(19) United States
(12) Patent Application Publication
(10) Pub. No.: US 2022/0062612 A1
(43) Pub. Date: Mar. 3, 2022

(54) FILL-PORT LOCATOR FOR A TISSUE EXPANDER

(71) Applicant: Edward Domanskis, Newport Beach, CA (US)

(72) Inventor: Edward Domanskis, Newport Beach, CA (US)

(21) Appl. No.: 16/873,959

(22) Filed: Aug. 31, 2020

(51) Int. Cl. A61M 39/02; A61B 90/00; A61F 2/12; A61M 39/04

(52) U.S. Cl. CPC A61M 39/0208; A61B 90/02; A61F 2/12; A61M 2039/0238; A61F 2230/0034; A61F 2210/009; A61M 39/04

(57) ABSTRACT

Disclosed is a locator for the fill-port of apparatus implanted under a patent's skin and connected by a flow tube to a breast-implanted tissue-expanding pouch. The embedded apparatus includes a circular metallic base and a dome-shaped self-sealing septum that is adapted for engagement by a syringe needle for injection of saline solution for filling the pouch. The locator is metallic and has a circular opening for engaging the patient's skin above the dome septum.

### BACKGROUND OF THE INVENTION

### 1. Field of the Invention

The present invention relates generally to breast implant surgery, and more particularly to a tissue expander pouch that is temporarily implanted within the breast, which pouch can be filled with saline solution in a gradual step-by-step process of stretching tissue to create a space for a larger permanent breast implant.

### 2. Description of the Prior Art

Tissue expanders are a type of a synthetic breast implant whose shell is a silicone elastomer. It is essentially a small pouch that will be expanded gradually over a period of weeks or months with saline injections. An access device is located under skin and tissue, commonly at a distance from the expander. The device, which has a flat metallic backing, features a fill port comprising a dome of self-sealing material that can handle multiple needle sticks.

In thin patients, the access device is easily located, however in the more obese patients in it sometimes difficult to palpate the port and may be time-consuming and requiring several attempts.

### BRIEF SUMMARY OF THE INVENTION

In view of the foregoing, it is a general object of the present invention to provide an improved way of filling a breast-implanted tissue expander.

One object is to minimize the amount of palpation required.

Another object is to allow greater accuracy of needle placement and to avoid multiple attempts,

A more particular object is to provide a quicker and easier way to locate the fill-port of an implanted access device for the tissue expander,

These and other objects and advantages are achievable by the present invention of an improved locator for the inlet-port for an implanted access device for a tissue expander, wherein the access device includes a fill-port comprising a dome-shaped self-sealing septum, and a circular, magnetized metallic base plate.

The locator comprises a generally flat steel ring having a flange portion and a central opening that is sized to snugly accommodate the tissue-covered septum of the implanted device, which may also make magnetic contact with base plate.

### BRIEF DESCRIPTION OF THE DRAWINGS

FIG. 1 is a perspective view of a preferred embodiment of a fill-port locator according to the present invention.

FIG. 2 is a side elevational view of the tissue expander access device used with the fill-port locator of the invention; and

FIG. 3 is a plan view of the locator of the invention positioned in place above the implanted access device.

### DETAILED DESCRIPTION OF THE INVENTION

Referring now to the drawings, FIG. 1 shows a fill-port locator 11 for use in conjunction with an access device 13 for a breast tissue expander, not shown. This tissue expander is a pouch of a suitable elastomer material that is surgically implanted within the breast. The access device 13 is also adapted to be surgically implanted, and spaced under the skin, or sometimes within the subcutaneous layer, at a short distance from the breast. It includes a circular metallic base plate 15 which may be magnetized. Among other things it will serve as a barrier to prevent/against penetration of a syringe needle.

Mounted to the top surface of the base plate 15 is a generally cylindrical fill-port housing 17 having an internal cavity that is capped by a dome-shaped self-sealing septum 19, which is preferably of a material that can tolerate multiple penetrations by the needle of a syringe.

A catheter-like polymeric tubing 21 is connected to the port housing and communicates with the housing cavity. The distal end of the tubing 21 connects to the tissue expander, not shown.

The fill-port locator 11, of FIG. 1, is constructed of stainless surgical steel, is flat and has a flange portion 23, and features circular opening 25. The diameter of the opening 25 is selected to slightly larger than the diameter of the device base 15, It should be appreciated that when the device 13 is in implanted position an amount of skin tissue will overlay the dome 19, creating a ‘dome’ of flesh tissue having an effective diameter which the opening 25 is sized to accommodate.

In using the locator 11, the physician or suitably trained person will manipulate the locator via flange 23 along the patient's skin surface to the vicinity of the implanted device, until it is centered over the device as illustrated in FIG. 3, and pressed downwardly in position, and held. The syringe operator will then insert the point of the syringe needle through the central region of the locator 11 and into the device dome 19 and inject a quantity of saline solution as required.

What is claimed is:

1. A fill-port locator for a tissue expander, which expander is surgically implanted within a patient's breast tissue, and which expander is connected by a saline solution flow tube that is implanted under skin tissue adjoining the breast, to an implanted fill-port device that includes a dome-shaped self-sealing septum having a maximum diameter and a circular metallic base plate, and wherein said locator comprises a generally flat metallic ring having a flange portion and a central opening that is larger than said septum diameter by a predetermined amount.

2. A fill-port locator as defined in claim 1 wherein said base plate is magnetized, and said locator is of a ferrous material.

3. A fill-port locator as defined in claim 2 wherein the dome of said implanted device presses outwardly against said skin tissue to form a dome of skin tissue, and said opening is adapted to accommodate said dome of tissue.

4. A fill-port locator as defined in claim 2 wherein said flange is adapted to be grasped by the fingers of an operator of said locator.

5. A fill-port locator as defined in claim 3 wherein said skin tissue covered septum dome has a maximum diameter, and said opening has a diameter appreciably larger than said skin tissue dome.
